(19) United States
(12) Patent Application Publication
(10) Pub. No.: US 2013/0123606 A1
(43) Pub. Date: May 16, 2013

(54) METHOD AND MAGNETIC RESONANCE APPARATUS TO MEASURE STRUCTURES OF THE HUMAN BRAIN

(71) Applicants: Bjoern Heismann, Erlangen (DE); Sebastian Schmidt, Weisendorf (DE)

(72) Inventors: Bjoern Heismann, Erlangen (DE); Sebastian Schmidt, Weisendorf (DE)

(21) Appl. No.: 13/661,246

(22) Filed: Oct. 26, 2012

(30) Foreign Application Priority Data
Oct. 28, 2011 (DE) 102011085404.5

(51) Int. Cl. A61B 5/00; A61B 5/055

(52) U.S. Cl. CPC A61B 5/4088; A61B 5/055; A61B 5/0042; A61B 5/0037; A61B 5/7225
USPC 600/410

(57) ABSTRACT

In a method to measure characteristic structure sizes of the human brain, which structure sizes can be used as biomarkers for a diagnosis of Alzheimer's, using a magnetic resonance device, manual and/or automatic localization of the hippocampus takes place in a preliminary magnetic resonance acquisition, followed by selection of at least one measurement axis that proceeds through the hippocampus. For each selected measurement axis, magnetic resonance data are acquired for a longitudinal, in particular rod-shaped, acquisition region proceeding along the measurement axis. Determination of the spatially resolved structure sizes is implemented from the magnetic resonance data.

### BACKGROUND OF THE INVENTION

1. Field of the Invention

The invention concerns a method to measure characteristic structure sizes of the human brain—which structure sizes can be used as biomarkers for an Alzheimer's diagnosis—using a magnetic resonance device. The invention also concerns a magnetic resonance apparatus designed to implement such a method.

2. Description of the Prior Art

Alzheimer's disease is a dementia illness frequently occurring in developed societies. Pharmaceutical development currently holds out the prospect of medicines or pharmaceuticals that markedly slow the course of the disease. Diagnostically, this means that the initial changes in the human brain due to this disease must be detected as early as possible so that the loss of cognitive capacity can be kept small. There are typically a few years between the occurrence of what is known as “mild cognitive impairment” (MCI, CDR0.5) and severe clinical symptoms (CDR3). The earlier that the illness is detected, the more brain function can be saved.

Various biomarkers are typically used to diagnose Alzheimer's, of which a significant one is known as brain atrophy (brain shrinkage), which is already detectable quite early (see for example the article “Multimodal techniques for diagnosis and prognosis of Alzheimer's disease” by Richard J. Perrin, Anne M. Fagan and David M. Holtzman, Nature 461, 916-922). It is known to use magnetic resonance devices to measure brain atrophy. High-resolution magnetic resonance sequences (MPRAGE, for example) and computationally intensive analysis means are used for this purpose, for example volumetry of brain areas with multivariant support vector machine evaluation. However, it has been shown that these techniques can be used only to a limited extent for the prediction of Alzheimer's. The primary problem is that the volumetric changes that have occurred to an extent so as to be detectable with conventional resolution already involve marked disease progression.

### SUMMARY OF THE INVENTION

An object of the present invention is to provide the ability to measure the human brain in a manner that delivers results that allow an earlier Alzheimer's diagnosis.

To achieve this object, in a method of the aforementioned type it is provided according to the invention that the following steps are used:

a) manual and/or automatic localization of the hippocampus in a preliminary magnetic resonance acquisition,

b) (in particular automatic) selection of at least one measurement axis that leads through the hippocampus,

c) for each selected measurement axis, acquisition of magnetic resonance data for a longitudinal—in particular rod-shaped—acquisition region traveling along the measurement axis,

d) (in particular automatic) determination of the spatially resolved structure sizes from the magnetic resonance (MR) data.

According to the invention, MR data are acquired from a longitudinal (in particular rod-shaped) acquisition region, ultimately to operate in essentially one-dimensional magnetic resonance imaging in order to determine the spatially resolved structure sizes. In such magnetic resonance sequences focused on one dimension, it is particularly advantageously possible to achieve high resolutions that can consequently deliver precise, spatially resolved structure sizes. This is particularly advantageous within the scope of the present invention since it in particular deals with brain atrophy, consequently the variation of thicknesses of defined brain regions that can be measured particularly well in one dimension. Values—for example the homogeneity of specific brain regions—can also be advantageously determined in such one-dimensional modes of observation.

For example, in an embodiment of the invention, at least one dimension of the hippocampus and/or at least one structure size describing the homogeneity of the hippocampus and/or at least one cortical thickness can be determined as the aforementioned structure size. In particular, occurrence of brain atrophy can be established under consideration of such structure sizes, wherein dimensions—in particular the thickness—of the hippocampus and the cortical thickness—thus the thickness of the cerebral cortex in comparison to typical sizes represent particularly suitable biomarkers. It should be noted that, if various measurement axes through the measurement axes are considered, the cerebral cortex is also inevitably acquired, such that cortical thicknesses thereof can be determined. However, it is also possible to determine, as the structure size, at least one dimension of at least one brain ventricle, but it is more difficult to place the measurement axes so that ventricles can be acquired.

To determine the structure sizes, initially different segments of the brain along the measurement axis are identified, in particular using a threshold method. Consequently, typical segmentation techniques can be used here in order to identify various segments of the brain (in particular the hippocampus and the cerebral cortex, possibly also ventricles) so that its dimensions (in particular the thickness) can be correspondingly determined, Because the attitude of the measurement axes is known, data from anatomical atlases and the like can also be taken into account to more precisely identify anatomical features in the brain.

In summary, in the method according to the invention, structure sizes related to concrete anatomical features of the brain (in particular the hippocampus and the cerebral cortex) are determined by using an essentially one-dimensional measurement method that allows a particularly high resolution along the measured dimension (thus along the measurement axis), without causing an excessively large extension of the data acquisition and/or a data flood that is due to longer evaluation times and more complex evaluation algorithms. In order to assess atrophying specifically in the cited field of Alzheimer's diagnosis (which deals with dimensions—ultimately thus thicknesses—of brain structures), such a one-dimensional measurement with high resolution offers advantages because, within the scope of a quite simple evaluation, the desired structure variables can nevertheless be determined. For example, the magnetic resonance data can be acquired with a resolution along the measurement axis of less than 200 μm, such that information in the range of 200 μm can be measured robustly. For example, at least 20 measurement points can typically exist in the hippocampus. The procedure according to the invention can be understood as a type of “virtual biopsy” that polls one-dimensional information.

In principle, such one-dimensional measurements of longitudinal—in particular rod-shaped—acquisition regions are known, for example within the scope of the measurement of what are known as navigators. Methods for high-resolution, one-dimensional measurements have also been described in the prior art, for example within the scope of an analysis known as fine structure analysis (FSA) that is offered by the company “Acuitas Medical”. However, there it is proposed to implement a non-spatially resolved evaluation in that a spatial frequency distribution—ultimately thus a seed size distribution—is determined via a Fourier transformation (see for example US 2007/0167717 A1).

In a more specific embodiment of the present invention, the acquisition of the magnetic resonance data takes place with a sequence in which two slices that are situated orthogonally to one another are excited, and the intersection region forms the acquisition region, in particular with a fine structure analysis sequence. In such an embodiment, excitation (for example within the scope of a spin echo sequence) of orthogonal slices takes place with the excitation pulses, for which the gradients are switched correspondingly. If two essentially cuboid slices are excited that are situated perpendicular to one another, the echo will ultimately arise in the intersection set of the two slices that is rod-shaped. The corresponding advantage is that the spatial resolution of the primary rod axis can be chosen to be very high, such that ultimately one-dimensional scans with a high spatial resolution are created. It has been shown that information in the range of approximately 200 μm can be robustly measured. In a further embodiment of the present invention, an automated segmentation method is used to localization the hippocampus. In this case as well, a segmentation method can thus be used as it is already known in principle in the prior art. Such segmentation methods can also be implemented under consideration of an anatomical atlas or the like.

At least three measurement axes can appropriately be selected which comprise an orthogonal trihedron. The use of an orthogonal trihedron of measurement axes lends itself to acquiring information in optimally many independent directions, for example so that thicknesses can be determined in directions that are orthogonal to one another and can subsequently be assessed. It is particularly advantageous to select at least four diagonals, covering all octants of the trihedron, as measurement axes. Additional useful information can be obtained in this way.

The acquisition of the magnetic resonance data can take place with a sequence emphasizing white and grey brain matter. A magnetic resonance sequence can thus be used that is optimized for the acquisition purpose and that in particular simplifies and aids the automatic evaluation. Corresponding methods that are applied to achieve a good contrast of the different brain matters are known in the prior art and thus need not be presented in detail herein.

Because the structure sizes have been obtained with the method according to the invention, an evaluation of the structure sizes with regard to the presence of Alzheimer's illness can take place as a following step in a method to diagnosis Alzheimer's. For example, for this purpose, it can be provided that the determined values for the structure sizes are compared with standard values that, for example, are stored in a database and/or can be determined from a plurality of acquired magnetic resonance images of healthy patients. However, it is particularly preferable for a comparison to be made with historical values acquired from the patient himself or herself, such that a temporal development can also be tracked, in particular a progressive atrophy and the like. A marked improvement of the early detection of Alzheimer's can be achieved in this way using the method according to the invention.

In addition to the method, the present invention also concerns a magnetic resonance device having a control device designed to implement the method according to the invention. All embodiments with regard to the method according to the invention can be achieved analogously to the magnetic resonance device according to the invention, with which the cited advantages can consequently also be achieved.

### BRIEF DESCRIPTION OF THE DRAWINGS

FIG. 1 is a flowchart of an embodiment, the method according to the invention.

FIG. 2 is a diagram illustrating the selection of measurement axes.

FIG. 3 is a diagram illustrating the acquisition of magnetic resonance data.

FIG. 4 is a diagram illustrating the determination of the structure sizes.

FIG. 5 schematically illustrates a magnetic resonance device according to the invention.

### DESCRIPTION OF THE PREFERRED EMBODIMENTS

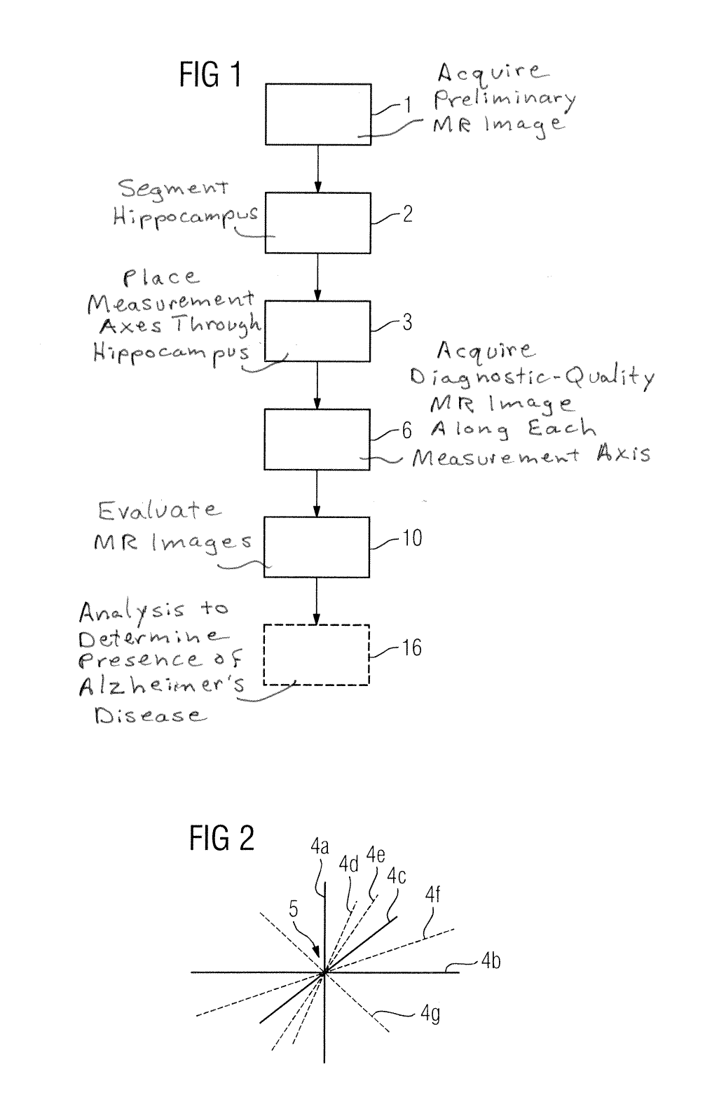

FIG. 1 shows a workflow plan of the method according to the invention in an exemplary embodiment. In Step 1, a preliminary magnetic resonance (MR) acquisition (in particular an overview image of the brain to be examined) is initially acquired there. Typical techniques to acquire such overview images can thereby be applied.

In the preliminary magnetic resonance acquisition, in Step 2 the hippocampus in the brain is automatically segmented (and thus localized) using segmentation algorithms known from the prior art.

Because the position and attitude of the hippocampus are now known, in Step 3 multiple measurement axes are automatically placed through the hippocampus (and consequently the entire brain) that should be measured in the following. However, here it is also conceivable that these measurement axes are manually established by an operator.

In the form of a principle drawing, FIG. 2 shows a possible selection of measurement axes 4a-4g that cover the space optimally well and deliver information in as many directions as possible. The measurement axes 4a, 4b and 4c clearly form an orthogonal trihedron 5, wherein the additional measurement axes 4d-4g are respectively diagonals through the octants that are defined by the trihedron 5.

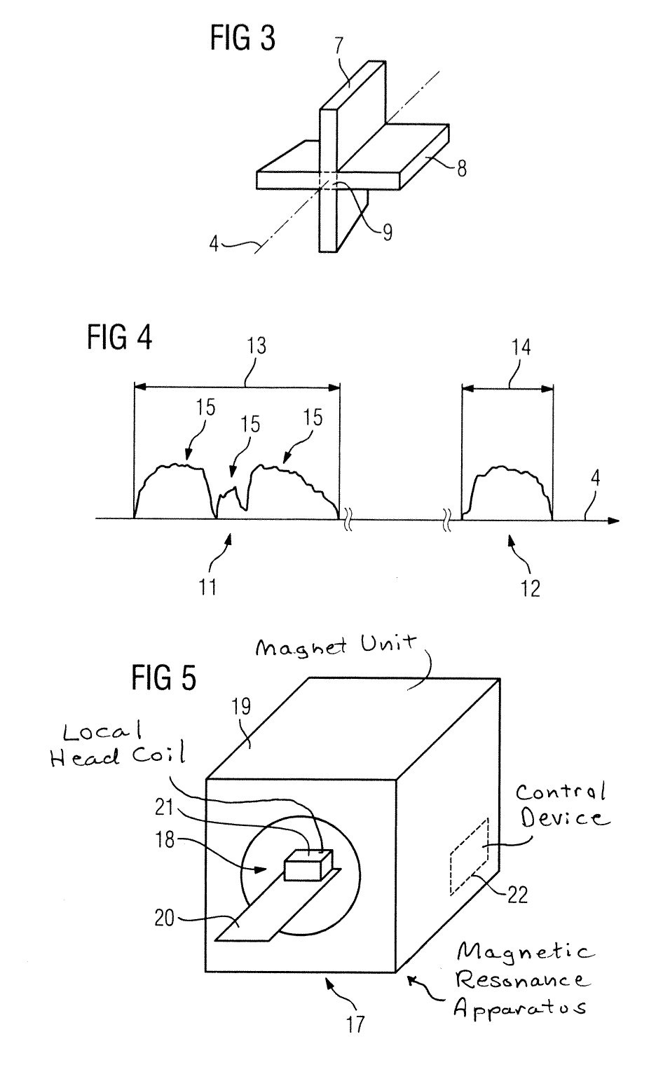

In Step 6, diagnostic-quality magnetic resonance data are then acquired for each of these measurement axes, more precisely one-dimensionally resolved magnetic resonance images of a rod along the corresponding measurement axis 4a-4g. For this a spin echo sequence is respectively used that is additionally designed for a particularly good contrast of white and grey brain matter. As is explained in detail by FIG. 3, in the spin echo sequence that is used different slices 7, 8 that are situated orthogonal to one another in the present exemplary embodiment are selected via corresponding switching of the gradients for the two excitation pulses. The slices 7, 8 are selected so that the longitudinal middle axis of their rod-shaped intersection region forms the measurement axis 4 that is to be directly measured. The acquisition region 9 in which nuclear spins are excited, and from which MR signals are obtained, is consequently the rod-shaped intersection region of the slices 7, 8.

The major rod axis consequently corresponds to the measurement axis 4 that is presently to be acquired, and the spatial resolution along the measurement axis 4 is selected to be very high, at present such that structures in the range of 200 μm can be measured. As has already been mentioned, a manner of one-dimensional magnetic resonance image is thus created whose pixel size in the direction of the measurement axis can correspond to 200 μm (preferably less than 200 μm), for example, such that high-resolution structures of he brain can be differentiated along the measurement axis.

As has already been mentioned, this acquisition of magnetic resonance data (thus the one-dimensional magnetic resonance images) takes place along each of the measurement axes 4 such that the hippocampus and the cerebral cortex are imaged.

The evaluation of the measurement data then takes in place in Step 10, in which (in the present exemplary embodiment) a measurement of the hippocampus, a structure size describing the homogeneity of the hippocampus, and a cortical thickness are determined as structure sizes for each of the measurement axes (in the cases of the cortical thickness da, where applicable). This is explained in detail by the principle drawing according to FIG. 4, which indicates a signal curve along the measurement axis 4 in the form of fictional magnetic resonance data. For example, the various anatomical subjects can be segmented automatically using threshold methods so that (for example) a hippocampus region 11 and a cerebral cortex region 12 are determined. The thickness or dimension 13 of the hippocampus and the cortical thickness 14 can now be read out in a particularly simple manner. Individual sub-structures 15 of the hippocampus can be evaluated to determine the structure size describing the homogeneity. The desired structure sizes can thus be determined automatically in a simple manner from the magnetic resonance data.

This enables an analysis with regard to a possible Alzheimer's disease to be implemented after an implementation of the method according to the invention as it is represented by Step 16 (indicated with dashed lines). For this purpose, the determined structure sizes can be compared with (for example) standard sizes stored in a database, or even to use older exposures (from which historical values for the structure sizes can be determined) for comparison. Due to the high resolution that is provided by the one-dimensional acquisition technique, variations can be detected particularly early, such that in particular an early detection of Alzheimer's is enabled by the method according to the invention.

FIG. 5 shows a schematic drawing of a magnetic resonance apparatus 17 according to the invention that—as is fundamentally known—has a basic magnet unit 19 defining a patient receptacle 18, wherein a radio-frequency transmission coil and a gradient coil arrangement are typically provided (not shown in detail) within the patient receptacle. A patient bed 20 that can be slid into the patient receptacle is provided, on which patient bed 20 can be arranged (as shown) a local head coil 21 that can be used particularly advantageously in the method according to the invention to acquire the magnetic resonance data.

The operation of the magnetic resonance device 17 is controlled by a control device 22 that is designed to implement the method according to the invention. In particular, all steps of the method according to the invention—in particular thus the calculations—can be implemented automatically.

Although modifications and changes may be suggested by those skilled in the art, it is the intention of the inventors to embody within the patent warranted hereon all changes and modifications as reasonably and properly come within the scope of their contributions to the art.

We claim as our invention:

1. A method to measure a characteristic size of an anatomical structure of a human brain, comprising:
operating a magnetic resonance (MR) data acquisition unit to obtain a preliminary magnetic resonance data set of a human brain in which the hippocampus is represented;
using a processor, localizing the hippocampus in said preliminary magnetic resonance data set;
using said processor, selecting at least one measurement axis that proceeds through the hippocampus;
for each selected measurement axis, operating said MR data acquisition unit to acquire diagnostic magnetic resonance data from a longitudinal acquisition region of the brain, said longitudinal acquisition region comprising a longitudinal extent along said measurement axis and having an extent perpendicular to said longitudinal extent that is substantially shorter than said longitudinal extent; and
using said processor, determining a spatially-resolved structural size of a structure represented in said MR data acquired from said acquisition region that is a biomarker for diagnosis of a presence of Alzheimer's disease.

2. A method as claimed in claim 1 comprising automatically, in said processor, localizing the hippocampus in said preliminary magnetic resonance data set.

3. A method as claimed in claim 1 comprising automatically, in said processor, selecting said at least one measurement axis that proceeds through the hippocampus.

4. A method as claimed in claim 1 comprising automatically, in said processor, determining said spatially resolved size of said structure.

5. A method as claimed in claim 1 comprising determining, as said size of said structure, a characteristic selected from the group consisting of at least one dimension of the hippocampus, at least one structural size describing a homogeneity of the hippocampus, and at least one cortical thickness of said brain.

6. A method as claimed in claim 1 comprising determining, as said size of said structure, at least one dimension of at least one ventricle in the brain.

7. A method as claimed in claim 1 comprising determining said size of said structure by, using said processor, identifying a plurality of different segments of the brain along said at least one measurement axis.

8. A method as claimed in claim 7 comprising identifying said plurality of different segments using a thresholding procedure.

9. A method as claimed in claim 1 comprising automatically localizing the hippocampus in said preliminary magnetic resonance data set using an automated segmentation algorithm.

10. A method as claimed in claim 1 comprising selecting at least three measurement axes proceeding through the hippocampus that define an orthogonal trihedron.

11. A method as claimed in claim 1 comprising acquiring at least said diagnostic magnetic resonance data by operating said MR data acquisition unit with a data acquisition sequence that emphasizes white and gray brain matter.

12. A method as claimed in claim 1 comprising acquiring at least said diagnostic magnetic resonance data by operating said MR data acquisition unit with a data acquisition sequence in which two slices that are orthogonal to each other are respectively excited, with an intersection region of said two slices forming said acquisition region.

13. A method as claimed in claim 12 comprising employing a fine structure analysis sequence as said data acquisition sequence.

14. A magnetic resonance (MR) apparatus comprising:
an MR data acquisition unit;
a control unit configured to operate said MR data acquisition unit to obtain a preliminary magnetic resonance data set of a human brain in which the hippocampus is represented;
a processor configured to localize the hippocampus in said preliminary magnetic resonance data set;
said processor being configured to select at least one measurement axis that proceeds through the hippocampus;
said control unit, for each selected measurement axis, being configured to operate said MR data acquisition unit to acquire diagnostic magnetic resonance data from a longitudinal acquisition region of the brain, said longitudinal acquisition region comprising a longitudinal extent along said measurement axis and having an extent perpendicular to said longitudinal extent that is substantially shorter than said longitudinal extent; and
said processor being configured to determine a spatially resolved structural size of a structure represented in said MR data acquired from said acquisition region that is a biomarker for diagnosis of a presence of Alzheimer's disease.
